(19) United States
(12) Patent Application Publication
(10) Pub. No.: US 2005/0038692 A1
(43) Pub. Date: Feb. 17, 2005

(54) System and method for facilitating centralized candidate selection and monitoring subject participation in clinical trial studies

(76) Inventors: John Michael Kane, Long Island City, NY (US); Paul Michael Gilbert, Skillman, NJ (US); Livingston Johnson, Skillman, NJ (US)

Correspondence Address: Hale and Dorr LLP, 650 College Road East, Princeton, NJ, 08540, US

(21) Appl. No.: 10/640,692

(22) Filed: Aug. 14, 2003

(51) Int. Cl. G06F 17/60

(52) U.S. Cl. 705/10

(57) ABSTRACT

A system and method of facilitating centralized and standardized remote ratings of subjects in clinical trial studies includes providing training to raters located at one or more central rating sites such that the raters are trained to apply substantially similar criteria in determining whether a candidate is a qualified subject for the clinical trial and/or in the actual assessment, or information collection, phase of the clinical trail. By having centralized, consistently trained raters that are independent of clinical trial investigators, inter-rater reliability is enhanced and potential bias is reduced, thus increasing the effectiveness of the clinical trial results by providing more qualified subjects and more accurate results.

### BACKGROUND OF THE INVENTION

The present invention relates to medical research business systems and methods; and more particularly, to systems and methods for facilitating the centralized and standardized assessments of subjects participating in clinical trials, particularly across large geographies.

High quality, rapidly executed clinical trials or studies are of great importance to the pharmaceutical industry. Each year, pharmaceutical and biotechnology companies spend billions of dollars on clinical research and development to develop new pharmaceutical compounds. Clinical trials are a vital, expensive and time-consuming part of the research and development process. It has been estimated that, for a given new drug, millions of dollars are lost for each day of delay in bringing the drug to market. These delays also prolong the time before new drugs or treatments are available to patients who need them. Since completion of clinical trials is almost always required before bringing a new drug to market, delays in execution and completion of such studies have a dramatic negative effect on timely availability of the drug as well as profitability. Moreover, delays in the studies lead to significantly greater costs associated with the study itself. Thus, the pharmaceutical and biotech industry have powerful economic incentives to conduct trials as quickly and effectively as possible.

Acceleration of clinical trials often results in poor selection of target population, the inclusion of patients who are inaccurately diagnosed or rated, who have had problems with response to or side effects from previous treatment, and who are not representative of the general population of patients with the disorder. The typical multisite studies used to complete enrollment of subjects for the trials quickly and lay the groundwork for broadly based market penetration are not informative concerning optimal use because most research sites have relatively small patient sample sizes. This also increases the likelihood of major monitoring difficulties, allowing greater outcome variations across sites. Thus, the attempt to increase power is foiled because sample sizes cannot be enlarged in studies that take place within a short period. (Kane, John M.D., “Improving Clinical Trials,” Arch Gen Psychiatry, Vol. 59 (March 2002), p. 273.

In spite of advancements in many aspects of clinical trial study design and execution, rapidly obtaining the participation, enrollment through completion of sufficient numbers of appropriate subjects for studies has remained the most significant barrier to rapid completion of high quality, cost-effective clinical trials. Typically subjects are obtained through advertisement campaigns or presentation or referral to sites actively involved in research recruitment. Subjects are not always paid. Subsequently, candidates are typically screened, interviewed or otherwise evaluated to screen out inappropriately qualified candidates and to obtain appropriate subjects for the study. The subjects then typically participate in the study by travelling periodically to clinical research sites for appropriate consultation, treatment, testing, assessment monitoring, data collection and the like, as required for the particular study. Typically, interested parties such as pharmaceutical companies contract with organizations such as contract research organizations, or CROs, to coordinate and manage the execution of studies.

In controlled clinical trials, establishing adequate reliability between different raters, or “inter-rater reliability,” is a major concern. Raters may be responsible for recruitment of qualified subjects as well as gathering the raw data with which the clinical trial is concerned. With regard to recruitment of subjects for a clinical trial, there is considerable difficulty in rapidly ascertaining appropriate patients since many, if not most patients, receive routine care in sites (i.e., a doctor's office) not involved in clinical research. Those medical sites which do participate in such trials are often primarily motivated by potential profit and are, therefore, often “stretching” to enter sufficient numbers of patients to generate adequate revenues in order to cover and exceed their fixed costs. As a result of being paid on a per patient enrolled basis, there is a natural inclination to err on the side of eligibility. Furthermore, the minimum threshold required by the FDA that a drug be superior to placebo, a lack of treatment or a specific comparative agent is more easily achieved if a larger sample size is used during the clinical trial, thus encouraging the recruitment of larger numbers of subjects.

Because of this over-enrollment tendency (i.e., including patients who are not technically eligible if assessments and ratings were done more accurately) it has been observed in some clinical trials, for example, that eligibility baseline scores for selected subjects tend to cluster at or slightly above the threshold entry criteria. This clustering can lead to an attenuating effect due to regression towards the means with respect to enrollment of those patients with borderline scores. Hence, lack of rigor in baseline assessments can impede the ability to detect the efficacy of an investigational agent (i.e., a novel chemical entity).

Furthermore, poor inter-rater reliability increases measurement error, which in turn increases the chance of Type II error, i.e., failing to detect true differences between an active drug and a placebo when such a difference does exist. For example, error, or variability, in the measurement of symptom severity is a significant contributor to uninterruptible results in multisite randomized clinical trials. Additionally, poor reliability decreases the statistical power of the clinical trial, resulting in the need for larger sample sizes to detect significant differences between the active drug and a placebo or between any active treatments or between treatments and control conditions of any type. For example, a study having an assessment reliability that falls from 1.0 to 0.80 drops in power from 0.80 to 0.71 and requires 25% more subjects to detect a significant difference (Muller & Szegidi, 2002). Given the time and financial resources involved in clinical trials (e.g., drug development), minimizing this source of error variance is of the utmost importance.

Establishing inter-rater reliability in multi-center clinical trials has been difficult for several reasons. For example, most antidepressant clinical trials use as their primary outcome measure clinician-administered symptom rating scales having ratings that are based on clinical judgment. However, scales such as the Hamilton Depression Rating Scale (HAMD; Hamilton, 1960; 1967), provide only general guidelines for the administration and scoring of specific items. No standardized questions or explicit scoring algorithms are provided. As a result, raters vary widely in how they administer the scale and score the items due to the subjective interpretation of the patents' statements, appearance, or behavior.

Most industry-sponsored trials do not adequately establish and assess inter-rater reliability within and across sites. Usually, a few videotaped interviews are screened at a rater's meeting (and frequently many of the sites' ultimate raters are not even present). No attempt is made to conduct formal statistical analysis of inter-rater reliability and few attempts are made to monitor inter-rater reliability on an ongoing basis. Even when inter-rater reliability is established, there is a drift in such reliability, since there can be a high turnover in raters and often raters are poorly supervised. Anything that can be done to reduce the number of different raters involved in a clinical trial, enhance their supervision, ensure their diligence in accurately assessing all relevant items and eliminating any potential incentives to bias ratings (e.g. in favor of eligibility) will enhance the quality and validity of clinical trials.

It is very telling that, when interested parties have asked investigators to submit audio tapes or videotapes of their assessments for review, despite the fact that these were “voluntarily” submitted, the results have often been very disappointing. Investigators often do not train raters to ask all of the appropriate questions or clarify answers in order to conduct an adequate interview and complete the rating scale in the correct, thorough manner.

For the most part, rater training at startup meetings has proven insufficient to obtain adequate results. For example, in one inter-rater reliability training effort for a multi-site clinical trial, the difference in maximum and minimum total HAMD scores (full scale range 0-52) (N=86 raters; 32 sites) evaluating the same subjects on videotaped presentations varied from a spread of 14 points in the best case, to a spread of 21 points in the worst case (Demitrack et al., 1997). The authors of the study concluded that measurement error is large, and that “there was no evidence of improved rating performance across the 6 hours of reliability training” (p. 20).

Methods and systems have been proposed for enhancing the accuracy of clinical trial studies, for example, co-pending U.S. patent application Ser. No. 10/076,738, entitled “System And Method For Facilitating Candidate And Subject Participation In Clinical Trial Studies,” filed Feb. 14, 2002, which is expressly incorporated herein by reference. None, however, alleviates the fundamental problem of ensuring inter-rater reliability and consistency in clinical trial studies. Thus, the above described problems in existing clinical trial studies persist.

### SUMMARY OF THE INVENTION

It is one feature and advantage of the present invention to conduct ratings in clinical trials using a small cadre of highly trained and well-monitored, centralized raters.

It is another feature and advantage of the present invention to enable raters to obtain centralized consent from trial subjects, thus ensuring that a full and complete consenting process is consistently implemented.

It is another feature and advantage of the present invention to provide consistency in training of raters, thus allowing for much stronger signal detection in, for example, the differences in the performance of a drug versus a placebo.

It is another feature and advantage of the present invention to provide centralized rater operations, thus disassociating raters from investigators and reducing economic incentives for raters to include subjects with borderline eligibility in a particular clinical trial just to meet some recruitment goal set by the investigators.

It is another feature and advantage of the present invention to conduct ratings in clinical trials at one or more core centers using remote communications methods, for example, the Internet, telemedicine and/or videoconferencing.

It is another feature and advantage of the present invention to reduce downtime of raters, and thus the overhead burden of conducting trials, by eliminating the need for raters to be available at local sites to conduct clinical trials.

It is another feature and advantage of the present invention to create better opportunities for blind or “masked” assessments of subjects in clinical trials.

It is another feature and advantage of the present invention to enhance quality control of clinical trials by recording (with appropriate consent) interviews for subsequent review.

These and other features and advantages of the present invention are achieved in a method for facilitating centralized and standardized ratings of subjects in clinical trial studies. The method includes training raters in at least one central rater site to employ substantially similar criteria for recruiting qualified subjects and/or collecting raw data from the qualified subjects in accordance with the clinical trial. The at least one central rater site may be at least one of a physical location or a virtual location. The method also includes interviewing qualified subjects by the raters located at the at least one central rater site. The interviewing may be performed to recruit qualified subjects and/or collect raw data from the qualified subjects on an ongoing basis according to the needs of the specific trial.

These and other features and advantages of the present invention also are achieved in a system for facilitating centralized and standardized ratings of subjects in clinical trial studies. The system includes at least one central rater site. Raters located at the at least one central rater site are trained to employ consistent criteria to recruit qualified subjects and/or to collect raw data from the qualified subjects. The at least one central rater site may be at least one of a physical location or a virtual location. The system also includes at least one satellite site at which study candidates are available to be interviewed by the centralized raters to determine whether the candidates are qualified subjects and/or to provide raw data to the raters.

These and other features and advantages of the present invention further are achieved in a computer usable medium storing program code which, when executed on a computerized device, causes the computerized device to execute a method for facilitating centralized and standardized ratings of subjects in clinical trial studies. The method includes training raters in at least one central rater site to employ substantially similar criteria for recruiting qualified subjects and/or collecting raw data from the qualified subjects in accordance with the clinical trial. The at least one central rater site may be at least one of a physical location or a virtual locations. The method also includes interviewing qualified subject by the raters located at the at least one central rater site. The interviewing may be performed to recruit qualified subjects and/or collect raw data from the qualified subjects.

Consistency in training of raters allows for much stronger signal detection in, for example, the differences in the performance of a drug versus a placebo. Consistency in training reduces the variability in the assessment strategies employed by raters and allows for more consistent and accurate results. Furthermore, centralizing rater operations and disassociating raters from investigators reduces economic incentives for raters to include subjects with borderline eligibility in a particular clinical trial just to meet some recruitment goal set by the investigators. By centralizing raters and segregating them from influence by the investigators, much more significant and accurate results can by obtained during the clinical trial.

There has thus been outlined, rather broadly, the more important features of the invention in order that the detailed description thereof that follows may be better understood, and in order that the present contribution to the art may be better appreciated. There are, of course, additional features of the invention that will be described hereinafter and which will form the subject matter of the claims appended hereto.

In this respect, before explaining at least one exemplary embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other advantages of the invention, along with the various features of novelty, which characterize the invention, are pointed out with particularity in the claims annexed to and forming a part of this disclosure. For a better understanding of the invention, and the specific advantages attained by its uses, reference should be had to the accompanying drawings and descriptive matter in which there is illustrated exemplary embodiments of the invention.

Other advantages of the present invention will be evident to those of ordinary skill, particularly upon consideration of the following detailed description of exemplary embodiments.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1A is a block diagram of a centralized system utilized in conducting a clinical trial study according to one or more embodiments of the present invention;

FIG. 1B is a block diagram of another centralized system utilized in conducting a clinical trial study according to one or more embodiments of the present invention;

FIG. 2 is a block diagram of a networked distributed computer system including central rater site computers, satellite site computers, interested party site computers, a clinical trial database and ancillary databases utilized in conducting a clinical trial study according to one exemplary embodiment of the invention;

FIG. 3 is a simplified depiction of a graphical user interface displayed on a central rater site computer monitor and an associated graphical user interface displayed on an associated satellite site computer monitor according to one exemplary embodiment of the invention;

FIG. 4 is a simplified depiction of one of the graphical user interfaces depicted in FIG. 3 and an associated set of technical equipment according to one exemplary embodiment of the invention; and

FIG. 5 is a flow diagram depicting a method for facilitating centralized candidate selection and monitoring subject participation in a clinical trial study according to one exemplary embodiment of the invention.

### DETAILED DESCRIPTION OF THE PRESENT INVENTION

In the following detailed description, numerous specific details are set forth regarding the system and method of the present invention and the environment in which the system and method may operate, etc., in order to provide a thorough understanding of the present invention. It will be apparent, however, to one skilled in the art that the present invention may be practiced without such specific details. In other instances, well-known components, structures and techniques have not been shown in detail to avoid unnecessarily obscuring the subject matter of the present invention. Moreover, various examples are provided to explain the operation of the present invention. It should be understood that these examples are exemplary. It is contemplated that there are other methods and systems that are within the scope of the present invention.

In addition, the following detailed description makes reference to the accompanying drawings that form a part hereof, and in which is shown by way of illustration a specific embodiment in which the invention may be practiced. It is to be understood that other embodiments may be utilized and structural changes may be made without departing from the scope of the present invention.

The present invention generally provides systems and methods for facilitating centralized and standardized ratings of subjects in clinical trial studies. Clinical trial patients in physician practices across geographies are assessed by remote investigative raters using, for example, video-conferencing, telemedicine platforms, and/or the Internet. The raters are highly trained in the use of subjective scales to minimize variability from rater to rater. Use of long-distance communication tools, such as video-conferencing, enables a high flow of patients and increases the economic justification of centralizing large groups of raters.

The phrase, “interested party” as used herein means any party or entity, such as, for example, an interested party of contract research organization, having an interest in a particular clinical trial study, such as having responsibility for the performance of the clinical trial study or having a need for the completion of the clinical trial study or the results of the clinical trial study.

The phrase “clinical trial study” as used herein is intended to broadly include many types of clinical or medical studies of subjects, generally in connection with obtaining data regarding the subjects' response to treatment such as, for example, the taking of medication. The term “medical professional” as used herein is intended to broadly include a wide range of individuals employed in medical or health care related fields, including, for example, physicians and nurses. The term “patient” (of a medical professional) as used herein is intended to broadly include any individual who has received or is receiving any medical or health care related treatment or advice by, through, or in connection with the associated medical professional. The term “pre-existing patient” as used herein means an individual who had been a patient of a medical professional prior to any recruitment or assistance in recruitment by the medical professional of the individual for a particular clinical trial study.

The term “candidates” is intended to broadly include individuals who are made aware of a clinical trial study so that they can consider participating as a subject, can attempt to participate as a subject, or can in fact participate as a subject in the study. The phrases “recruitment of subjects”, “recruitment . . . as subjects”, and the like as used herein refer broadly to conduct intended to lead to obtaining candidates or subjects for a study. For example, “recruitment of subjects” includes making an individual who may potentially be suitable for participation in a clinical trial study aware of the clinical trial study, recommending that the individual consider participating or attempting to participate in the study, or actually enrolling the individual as a subject in the study. The term “investigator” as used herein refers to any individual or group with substantial responsibility in the performance of a clinical trial study, including, for example, the coordination or management of a clinical trial study, or analysis of clinical trial study data, and including, for example, medical professionals involved with the study.

The term “rater” as used herein refers to any individual or group with substantial responsibility in choosing acceptable subjects for a clinical trial, making subjective and/or objective assessment of a subject's condition that is under study by interviewing potential subjects of the clinical trial, rating those potential subjects, and/or collecting raw data regarding the product(s) under test by interviewing acceptable subjects over the course of the trial. A rater may be a medical professional, e.g., doctors, nurses, etc., non-medical professionals, e.g., social workers, or non-professional trained on rating procedures and methodology.

The terms “rating” and “ratings” as used herein refer to, but are not limited to, one or more of subject ascertainment, selection, recruitment, consenting, assessment, and monitoring in clinical trials.

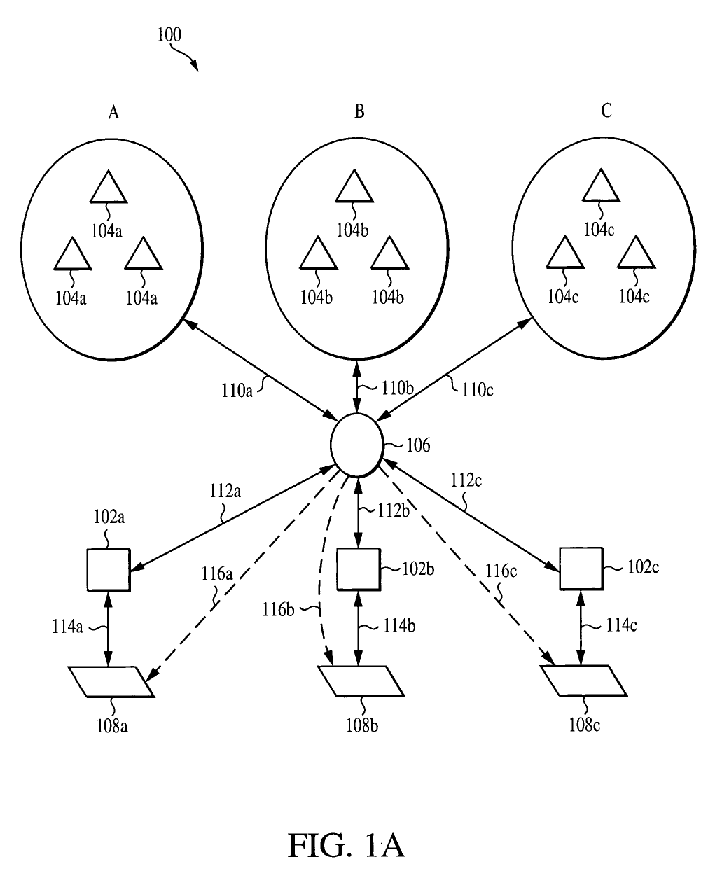

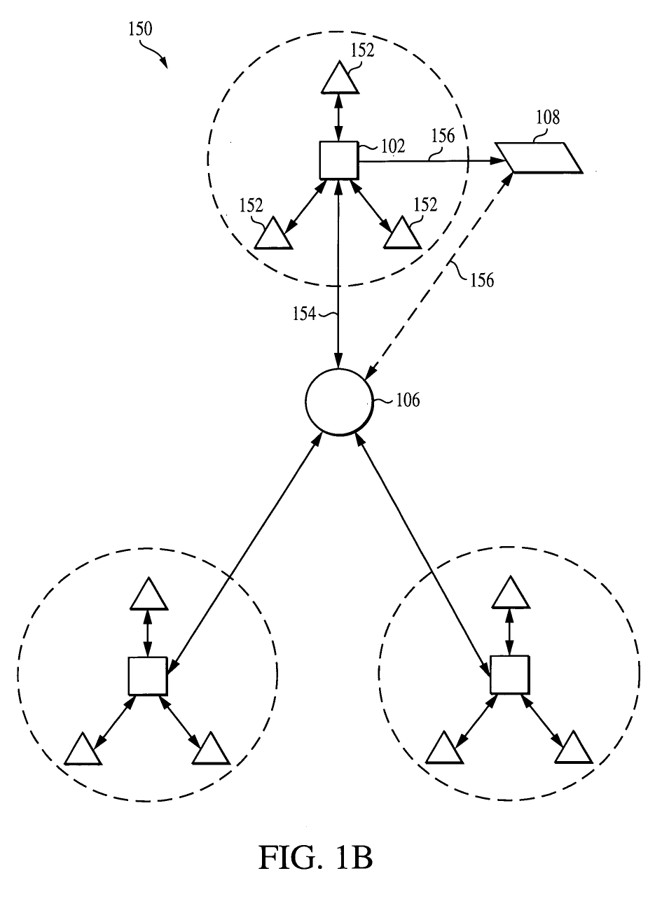

FIG. 1A is a block diagram of a system 100 utilized in conducting a clinical trial study according to one exemplary embodiment of the invention. System 100 includes, for example: one or more investigators 102; one or more candidates 104; one or more interested parties 108; and at least one central rater site 106. Interested parties 108 may instruct investigators 102 to conduct a clinical trial for a new product, e.g., a new drug. Alternatively, investigators 102 may initiate a clinical trial on their own. Subjects for the clinical trial are drawn from a candidate pool 104. Although one investigator 102 is shown, investigator 102 may also include one or more primary investigators and one or more respective sub-investigators under the control and direction of the primary investigator for a particular clinical trial scenario. Individuals in candidate pool 104 are, for example, patients in doctor's offices and/or clinics. Investigators solicit independent, expert raters at central rater site 106 to, for example, select the appropriate candidates 104 based on some selection criteria and to interview selected candidates 104 to obtain the raw data of the clinical trial.

Raters might also conduct diagnostic interviews, inform patients about the nature of the research, e.g., the risks and benefits, and participate in the process of obtaining informed, centralized consent. Implementing centralized informed consenting of the patients ensures that the consenter, for example, the centralized rater(s), is executing a full and complete consenting process. Frequently, due to lack of time, physicians may not take patients through the full consenting process and may skim or skip over sections of the consenting form. Patients may also feel somewhat pressured to consent because their physician may appear to wish that they consent. Removing the consenting from the physician, who may be economically motivated to have the patient participate in the trial, and putting the consenting process in the hands of independent consenters (e.g., raters) eliminates these issues.

Raters might also interview and assess family members or significant others with regard to the patient's condition, course over time, response to treatment, and level of functioning. Raters may also assess the care giver, relative, or significant other on the effects of the patient's condition, on their own mental state, subjective burden, quality of life, or functioning. The rater may also assess the patient for adverse effects of treatments being received. Raters (or their centralized staff) may be available to answer questions about the patient's illness, the study, or other matters relevant to facilitating the ongoing participation of the patient in the trial. Candidates 104 may be situated in various locations, for example, a doctor's office, a hospital, a clinic, an office, school, house of worship, factory floor, or any community location. Raters at central rater site 106 may also be solicited directly by interested parties 108. Central rater site 106 may be a single physical location. Furthermore, although one central rater site 106 is illustrated, several such central rater sites 106 may be utilized. Also, central rater site 106 may be virtual, such that the actual raters are not located in any specific geographic location(s).

Raters in the at least one central rater location 106 are subjected to similar training and preparation, such that the same criteria and standards are used in the selection and subsequent interviewing of candidates 104. Raters at central rater location(s) 106 may alternatively be kept blind to such factors as selection criteria, study design of the clinical trial, visit number, etc., thus enhancing assay sensitivity. Assay sensitivity is the ability of a clinical trial to discern a clear benefit for the product being tested, when there is a benefit. Additionally, centralizing raters, rather than having raters associated with a particular investigator, removes any incentives for raters to inflate baseline scores or to recruit non-qualified patients or to exaggerate change scores, minimize side effect scores or to introduce any subjective bias because of economic incentives offered implicitly or explicitly by the investigator.

FIG. 1A illustrates an example of centralized system 100. In this example, three different clinical trials are being conducted: trial A; trial B; and trial C. The individual trials may be initiated by separate interested parties 108a, 108b, and 108c. The interested parties 108a, 108b, 108c may then instruct a particular investigator 102a, 102b, and 102c, respectively, to conduct the clinical trial. It is also possible that more than one investigator 102 may be contacted by a single interested party 108.

Each trial A, B, and C may have separate pools of candidates. For example, trial A may be focused towards candidate pool 104a, which is, for instance, a target age group. Trial B may be focused towards candidate pool 104b, which is, for instance, a target gender group. Trial C may be focused towards candidate pool 104c, which is, for instance, a target ethnic group. Of course, individual patients may fall into more than one candidate pool.

For trial A, for example, investigator 102a, or alternatively, interested party 108a, utilizes raters at central rater site 106 to screen candidate pool 104a to determine which individuals in candidate pool 104a are qualified to become subjects for trial A through appropriate screening and selection techniques. Raters at central rater site 106 are trained such that the raters apply consistent screening and selection procedures for all potential subjects of the clinical trial. After selecting appropriate subjects from candidate pool 104a, raters at central rater site 106 conduct interviews with subjects using remote communication method 110a. For example, the interviews may be done over the telephone, may be done using video conferencing, may be done over the Internet, or any other suitable communication medium that allows the rater(s) to interact with the candidates and/or subjects. The raw data collected during the interviewing process is then forwarded to investigator 102a (112a), who may then process and analyze the raw data or, in turn, forward the raw data to interested party 108a for processing and analysis. Alternatively, the raw data may be forwarded directly to interested party 108a (116a) for processing and analysis.

A similar scenario takes place for trial B and trial C. Again, more than one central rater site 106 may be used for any one trial, provided that there is consistency in the training of raters and criteria applied by raters in selecting appropriate subjects from the candidate pools in all central rater sites.

FIG. 1B illustrates another example of a centralized system for centralizing clinical trials. Centralized system 150 utilizes a hub-and-spoke system in which one or more “hubs” of investigators 102 interacts with one or more “spokes” of satellite sites 152, respectively. Satellite sites 152 can be a doctor's office, a clinic, a subject's home, an office building, a school, a community center, etc. Clinical trial study coordination, management, and management of satellite locations 152 is performed by investigators 102. Raters, who are involved with the recruitment, etc. of candidates operate at central rater site 106, which acts as a virtual “super-hub.” Each group of investigators 102 and satellite sites 152 may be involved in the same or different clinical trial activities. However, only raters associated with the central rater site 106 conduct rating activities. Again, central rater site 106 may be one or more physical locations or can be a truly virtual site, such that raters are geographically disparate. However, the raters associated with central rater site 106 have similar training and assessment skills and apply similar assessment criteria, as discussed previously.

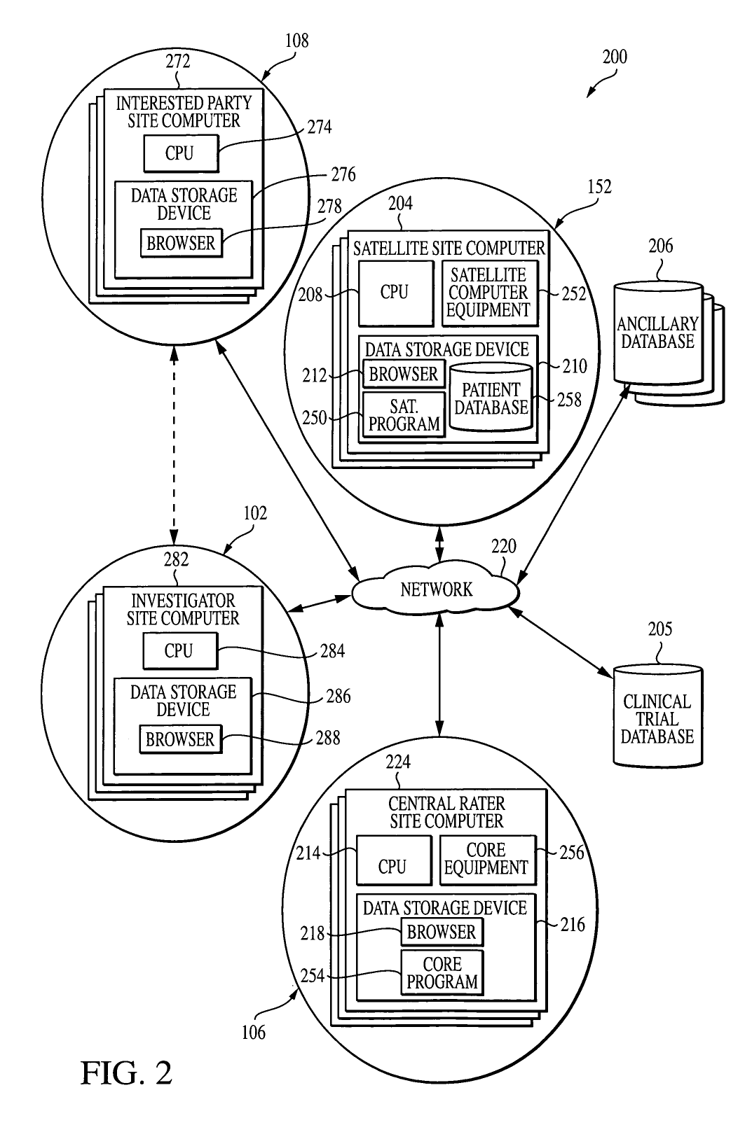

FIG. 2 depicts a block diagram of a networked distributed computer system 200 utilized in conducting a centralized clinical trial study according to one exemplary embodiment of the invention. The computer system 200 includes, for example: one or more central rater site computers 224 located at central rater site 106; one or more satellite site computers 204, each set of the satellite site computers 204 being located at a different satellite site 152; one or more interested party site computers 272 located at interested party site 108; one or more investigator site computers 282 located at investigator site 102; a clinical trial database 205, and one or more ancillary databases 206. The network 220, connecting the various computers 204, 224, 272, 282 and the databases 204, 206, is intended to broadly include, but is not limited to, any of various types of computer networks or an array of networks which can include one or more local area networks, one or more wide area networks, such as the Internet, an intranet, satellite, and telephonic communication means. In addition, the network 220 can be a wireless network, and communication between computers can be through wireless connections, such as, for example, wireless Internet connections. Furthermore, network 220 may include other medium of transmission, for example, a T-1 line.

Each of the satellite site computers 204, the central rater site computer 224, the interested party site computer 272, and the investigator site computer 282 includes, for example, one or more central processing units (CPUs) 208, 214, 274, 284 and one or more data storage devices 210, 216, 276, 286 comprising one or more browser programs 212, 218, 278, 288 respectively, to allow access to and communication through the network 220. For example, in embodiments in which the network 220 is the Internet, the browser programs 212, 218, 278, 288 can be Microsoft's Internet Explorer or another Internet browser. The data storage devices 210, 216, 276, 286 may include various amounts of RAM for storing computer programs and other data. In addition, the central rater site computer 224, satellite site computers 204, interested party site computer 272, and investigator site computer 282 may include other components typically found in computers, including one or more output devices such as monitors, other fixed or removable data storage devices such as hard disks, floppy disk drives and CD-ROM drives, and one or more input devices, such as mouse pointing devices, styluses, cameras, and keyboards. In addition, various other computer and computer related components may be utilized, in part, to enhance communication between the central rater site computer 224 and the satellite site computers 204.

Generally, the central rater site computer 224, the satellite site computers 204, and the interested party site computer 272 operate under and execute computer programs under the control of an operating system, such as Windows, Macintosh, UNIX, etc. Further, generally, the computer programs of the present invention are tangibly embodied in a computer-readable medium, e.g., one or more data storage devices attached to a computer. Under the control of an operating system, computer programs may be loaded from data storage devices into computer RAM for subsequent execution by the CPU. The computer programs include instructions which, when read and executed by the computer, cause the computer to perform the steps necessary to execute elements of the present invention.

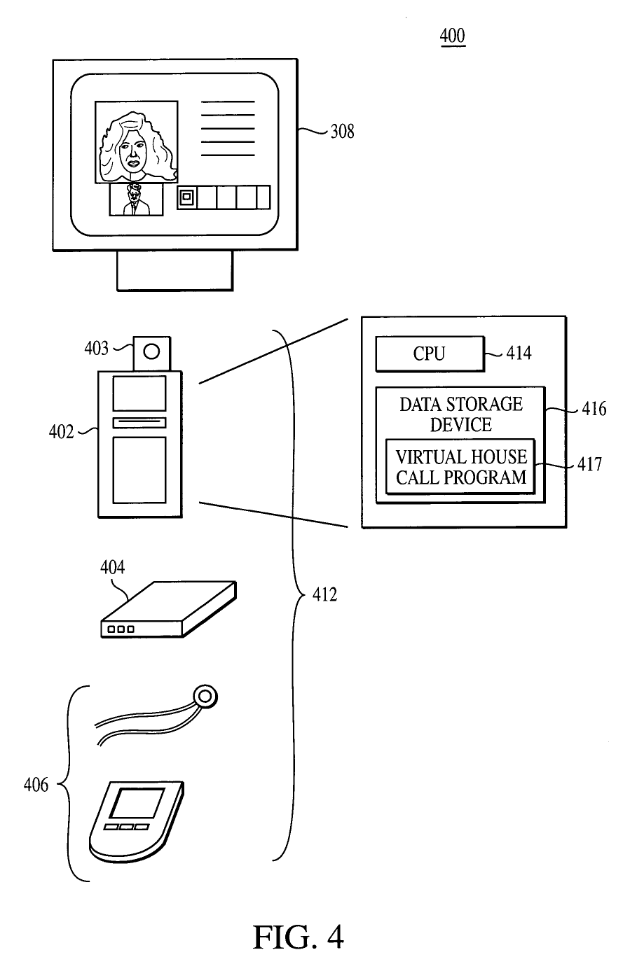

The satellite site computers 204 include satellite computer equipment 252, and the data storage devices 210 of the satellite site computers include a satellite program 250. The satellite site computers 204 are programmed and equipped to allow medical professional and subject participation from the satellite sites 152, and communication of clinical trial data obtained from the subjects to the central rater site computer 224. One exemplary embodiment of the satellite computer equipment 252 and satellite program 250 are described in detail with reference to FIG. 4.

Central rater site computer 224 includes core computer equipment 256, and data storage device 216 of central rater site computer 224 includes core program 254. The core computer equipment 256 and the core program 254 include all the equipment and programming necessary to support central rater site functions, including communication and interfacing with the satellite site computers 204 as well as study coordination. Collected clinical trial study data is ultimately sent over the network to the interested party site computer 272, where it is analyzed in accordance with the study. For example, in various embodiments of the invention, collected clinical trial study data can be sent from the satellite site computers 204 simultaneously to both the central rater site computer 224 and the interested party site computer 272, or the clinical trial data can be sent from the satellite site computers 204 to the central rater site computer 224 and from the central rater site computer 224 to the interested party site computer 272. In addition, in some embodiments of the invention, collected clinical trial study data can be sent from the satellite site computers 204 to some intermediary source, and sent from the intermediary source to the central rater site computer 224.

In some embodiments, clinical trial data is collected from subjects at the satellite sites 152, input into the satellite site computers 204, and communicated over the network 220 to the central rater site computer 224 using, for example, video, audio, manual input (i.e., with a keyboard), etc. Clinical trial data may be nonvolatilely stored in the data storage devices 216, 210 of the central rater site computer 224 or the satellite site computers 204, respectively, in the clinical trial database 205, or any combination thereof. For example, clinical trial data may be input into the satellite site computer 204 and immediately communicated over the network 220 to the central rater site computer 224 for nonvolatile storage therein, or may be communicated to the clinical trial database 205 and accessed or manipulated remotely from the central rater site computer 224. Although only one clinical trial database 205 is depicted, in other embodiments, multiple clinical trial databases in one or more locations are utilized. Information from the clinical trial database 205 can be used and analyzed in various ways to complete the objectives of the study, and, in some embodiments, for other purposes as well, as explained further below.

The databases 204, 206, 258 may include, for example, any of numerous types of databases, including, for example, an Oracle® relational database system, commercially available from Oracle® Corporation, a commercially available DB2 database, a Lotus® Domino™ server computer database, a Sybase® database, available from Sybase® Corporation, Microsoft® Structured Query Language (SQL) servers, and various Open DataBase Compliant (ODBC) databases.

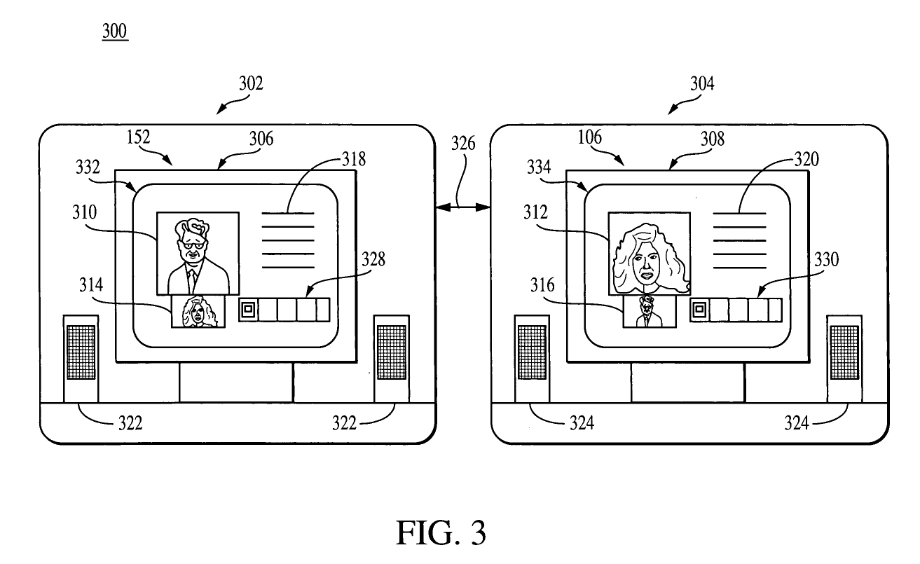

FIG. 3 is a simplified depiction of a graphical user interface 334 displayed on a central rater site computer monitor 308 at a central rater site 106 and an associated a graphical user interface 332 displayed on an associated satellite site computer monitor 306 at a satellite site 152 according to one exemplary embodiment of the invention. In some embodiments of the invention, real-time or almost real-time video and/or audio, on-line chat, or other communications technologies, such as video-conferencing, are utilized to enhance communications between central rater sites 106 and satellite sites 152.

Further, in some embodiments of the invention, software, as well as hardware or other equipment, is utilized in providing enhanced communications relating to the clinical trial study between central rater site computers 224 and satellite site computers 204. FIG. 3 is intended to help illustrate an embodiment in which real-time or almost real-time video is utilized in communications between the central rater site computer 224 at a central rater site 106 and a satellite site computer 204 at a satellite site 152, and in which software is utilized to further enhance or simplify the experience for users. FIG. 3 includes a simplified snapshot 302 of the satellite site computer area and a simplified snapshot 304 of the central rater site computer area. Double-headed arrow 326 is intended to indicate that the central rater site computer 224 as well as the satellite site computer 204 are both connected to the network 220, e.g., the Internet, and communicate with each other utilizing, for example, high-speed Internet access connections, such as cable modems or DSL.

As depicted in FIG. 3, the graphical user interfaces 332, 334 are custom provided utilizing software, which can be programmed by one skilled in the art based on the description provided herein, to suit anticipated needs of a participating subject using the satellite site computer 204 at a satellite site 152 communicating with, for example, a medical professional or an investigator using the central rater site computer 224 at a central rater site 106. Specifically, FIG. 3 depicts an example of a real-time or almost real-time two-way video and audio conference between a subject using a satellite site computer 204 located at a satellite site 152 and a rater using a central rater site computer 224 located at a central rater site 106. At the satellite site computer monitor 306, a window 310 shows an image of the rater engaged in the conference. Small window 314 shows an image of the subject who is sitting in front of the monitor 306. Similarly, at the central rater site computer monitor 308, a window 312 shows an image of the subject engaged in the conference. Small window 316 shows an image of the rater who is sitting in front of the monitor 308. Sets 322, 324 of speakers at each of the computers 204, 224 are used in providing real-time or almost real-time audio conferencing capability.

Each of the graphical user interfaces 332, 334 may also include text areas 318, 320 with lines of text, which is shown in simplified form as straight lines. The text areas 318, 320, as depicted, are used for on-line chatting between the subject and the rater, but may be used for other purposes in various embodiments, such as to show medical or health care information, or a passage from an on-line article or to list questions or items included in the assessment process.

The graphical user interfaces 332, 334 also include multiple button tool bars 328, 330. Special software programs can be executed to facilitate use of buttons on the tool bars 328, 330 to aid in the conference, which may be a “virtual house call” in which the subject communicates from the satellite site computer 204 clinical trial data or other medical data to the rater at the central rater site computer 224, and the rater interacts with the subject to ask questions relating to the subject's suitability for the clinical trial and/or substantive questions relating to clinical trial, itself. For example, the buttons may each be used to represent a particular medical monitoring instrument, and pressing the button may cause a new window to open displaying detailed information about current or past readings from the instrument, or the new window may show the instrument itself showing the readings. In some embodiments, the screens of the monitors 306, 308 are touch sensitive, and a pen-like device, or stylus, may be used to select buttons or otherwise interact with the graphical user interfaces 332, 334. In other embodiments, a pointing device such as a “mouse”, or simply a keyboard may be used. In some embodiments, users participate in virtual house calls even though they have a minimum of computer proficiency.

Although one or more embodiments of the present invention may utilize computerize video-conferencing as described above, other methods of communication between the subject(s) and rater(s) are contemplated. For example, communication may be done using conventional video-conferencing methods without the use of a computer or using conventional telephonic means. Furthermore, conferencing done using a computer may be performed without the use of a visual component and/or an audio component and may be text only, e.g., a live on-line chat. Other, non-real-time methods of computerized communication may also be utilized, such as electronic mail.

FIG. 4 is a simplified depiction of one of the computer monitor 308 depicted in FIG. 3 and an associated set 412 of technical equipment that can be utilized in or as part of some embodiments of the satellite site computer 204, and in implementing the virtual house call as discussed with reference to FIG. 3 above. The equipment and programming described with reference to FIG. 4 represents one exemplary embodiment of the satellite site computer 204, including the satellite site computer equipment 252 and the satellite site computer program 250.

As shown, the set 412 includes, for example: a control station computer 402; a camera 403 for use with a computer, or “Webcam”; a cable modem 404, or a router, or other such device that allows satellite site computer 204 to connect to network 220; and one or more electronic medical monitoring devices 406. The camera 403 is used in providing video conferencing functionality. The cable modem or router 404 provides high-speed Internet access, such as, for example, Internet access at a download speed of 100,000 bits per second or higher.

The one or more electronic medical monitoring devices 406 may include, for example, a stethoscope, a pulse Oximetry monitor, a thermometer, a weight scale, a blood pressure monitor, and other devices to provide clinical trial data and other medical or health care data from subjects or other participants. In the embodiment depicted, the electronic medical monitoring devices is connected to the control station computer 402 and information from them can be displayed or interacted with using the graphical user interfaces 332, 334 (as shown in FIG. 3). In some embodiments, the set 412 of equipment is utilized in acquiring clinical trial data from subjects at satellite sites 152, in some cases with assistance from medical professionals, and in communicating the data to central rater site(s) 106. The set of equipment may also include other electronic devices, such as personal digital assistants (PDAs) which can connect to and operate in cooperation with a computer, and which may aid a medical professional participating in a study or a subject in activities they perform in connection with the study.

The control station computer 402 includes, for example, a CPU 414 and a data storage device 416. The control station computer 402 is utilized in some embodiments of the invention in which the central rater site computer 224 or satellite site computer 204, along with various other equipment for use with the computers, are specially provided for the clinical trial study. In some embodiments, the control station computer 402 and associated peripheral equipment may be one of the central rater site computers 224 or one of the satellite site computers 204, or, in other embodiments, it may be part thereof.

In one or more embodiments of the present invention, the data storage device 416 includes, for example, a virtual house call program 417. The virtual house call program 417 is intended to broadly represent all programming necessary to carry out computer functions appropriate in carrying out clinical trial study related activities, such as, in some embodiments, virtual house calls, as described above with reference to FIG. 3. The exact configuration of the satellite site computers 204, and, if utilized, the control station computer 402 will vary depending on the exact requirements of the particular clinical trial study for which they are being used. Similarly, the virtual house call program 417, or other software used to provide clinical trial study related uses, will also vary. In some embodiments, the electronic virtual call program 417 helps allow users to participate in such conferences with a minimum of computer proficiency or experience with using the virtual house call program 417.

While FIG. 4 describes satellite site computer equipment and programming, it is to be understood that the central rater site computer 224 may also include, among other things, all the necessary hardware and programming to support interface with the satellite site computers 204.

It should be understood that, in different embodiments of the invention, the central rater site computer 224 and satellite site computers 204 may be computers that were already present prior to any clinical trial studies and may have been used for general office purposes, for example. In some embodiments, such computers can be upgraded, additionally equipped, provided with additional software, or provided with Internet access or faster Internet access, to allow them to be used in a particular clinical trial study in accordance with the invention. In other embodiments, the computers 204, 224, software, equipment, and Internet access can be designed or provided specially or exclusively for use in the clinical trial study.

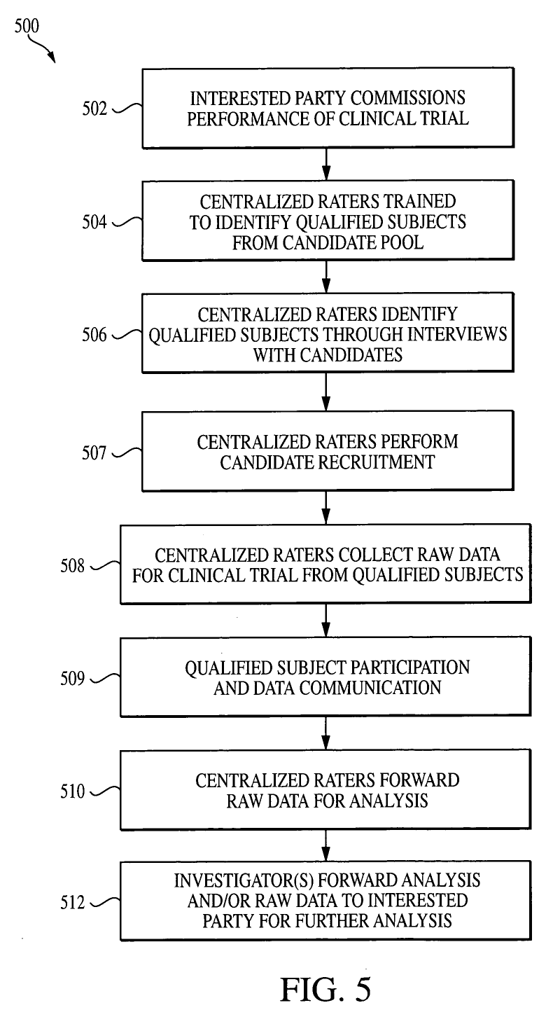

FIG. 5 is a flowchart illustrating a method 500 for facilitating centralized and standardized ratings of subjects in a clinical trial study according to one or more embodiments of the present invention. Method 500 may be used, for example, in conjunction with the system model of FIG. 1A. An interested party 108 initiates the process by commissioning performance of a clinical trial (step 502). Interested party 108 may commission one or more investigators 102 directly to perform the clinical trial. However, it should be noted that in some embodiments, the clinical trial can be performed without the use of separate investigators. In such cases, the raters, themselves, may perform any functions typically associated with investigators. The interested party may be, for example, a pharmaceutical company, a biotechnology company, etc. Raters at central rater site 106 are then trained, for example, by investigator(s) 102, or some other entity, to identify qualified subjects from candidate pool(s) 104 (step 504). As discussed previously, raters at central rater site 106 receive consistent training and employ consistent selection criteria such that inter-rater reliability is increased.

Raters at central rater site 106 identify qualified subjects for the clinical trial (step 506) through interviews with candidates. These interviews may be performed using the means described with respect to FIGS. 3 and 4, with candidates communicating with the raters from, for example, satellite site computer(s) 204. The raters, in turn, may communicate with candidates using central rater site computer(s) 224.

At step 507, candidate recruitment is performed by raters at the central rater site(s) 106. Step 507 may include any necessary screening of candidates by the raters to make sure they are sufficiently qualified to participate in the study, such as by being the appropriate age, having the appropriate medical condition, etc.

In some embodiments, step 507 includes obtaining data from candidates, such as candidates who indicate that they may wish to participate in future studies, and storing the data in an ancillary database, to be utilized in future study planning and recruitment activities. Also, step 507 can include utilizing information of this type from previous studies in order to identify, target, or approach potential candidates for the study. Step 507 also includes inviting qualified candidates to participate as subjects, and obtaining any necessary agreements or consents, including written agreements or consents, from candidates who agree to participate as subjects. Step 507 is also intended to include any recruitment, which may need to be performed at a later time to replace any subjects who unexpectedly decide to not participate or who drop out but can be replaced. As such, step 507 can be revisited at different stages in the method 600.

Subsequent to identifying qualified subjects, rater(s) may then conduct assessment interviews with the qualified subjects and collect the desired raw data for the clinical trial (step 508). Again, the assessment interviews may also be performed using the means described with respect to FIGS. 3 and 4. At step 509, qualified subjects participate in the study and data is obtained and communicated in accordance with the invention. Step 509 includes communicating clinical trial data from satellite sites to the central rater site(s) in accordance with the study. Step 509 also includes, in some embodiments of the invention, monitoring by the integration organization of study conduct and execution.

After the assessment interviews are concluded, the raters at central rater site(s) 106 forward the raw data to the investigator(s) 102 for analysis (step 510). The investigator(s) 102 may then, in turn, forward the analysis and/or the raw data, itself, to the interested party 108 for further analysis in conjunction with the clinical trial (step 512). Alternatively, the raters may forward the raw data directly to interested party 108 for analysis.

While the invention has been described and illustrated in connection with exemplary embodiments, many variations and modifications as will be evident to those skilled in this art may be made without departing from the spirit and scope of the invention, and the invention is thus not to be limited to the precise details of methodology or construction set forth above as such variations and modification are intended to be included within the scope of the invention.

1. A method for facilitating centralized and standardized ratings of subjects in clinical trial studies, the method comprising:
training raters located at at least one central rater site to employ substantially similar criteria for at least one of recruiting qualified subjects and collecting information from the qualified subjects; and
interviewing the qualified subjects by the raters located at the at least one central rater site, wherein the interviewing is to accomplish the at least one of recruiting qualified subjects and collecting information from the qualified subjects.

2. The method of claim 1, wherein the raters obtain centralized informed consent from the qualified subjects.

3. The method of claim 1, wherein the at least one central rater site is at least one of remote and centralized from the subjects.

4. The method of claim 1, wherein the at least one central rater site is at least one of a similar physical location or a virtual location.

5. The method of claim 1, wherein the qualified subjects are located at at least one satellite location.

6. The method of claim 5, wherein the at least one satellite location comprises at least one of a doctor's office, a clinic, a qualified subject's home, a hospital, a school, an office building, a shopping mall, a house of worship, and a factory.

7. The method of claim 1, wherein the interviewing is accomplished utilizing virtual communications over a network.

8. The method of claim 7, wherein the network comprises the Internet.

9. The method of claim 7, wherein the virtual communications comprises at least one of a video and audio conference, a live on-line chat, and electronic mail.

10. The method of claim 1, wherein the step of interviewing is accomplished utilizing video-conferencing.

11. The method of claim 1, wherein the step of interviewing is accomplished utilizing at least one of audio and video telephonic means.

12. The method of claim 1, further comprising the step of forwarding the collected information to at least one investigator for analysis.

13. The method of claim 12, wherein the step of forwarding comprises the step of forwarding at least one of the collected information and an analysis of the collected information to at least one interested party.

14. The method of claim 1, further comprising the step of forwarding the collected information to at least one interested party for analysis.

15. A system for facilitating centralized and standardized ratings of subjects in clinical trial studies, the system comprising:
at least one central rater site comprising raters that are trained to employ consistent criteria for at least one of recruiting qualified subjects and collecting information from the qualified subjects; and
at least one satellite site, coupled to the at least one central rater site, at which study candidates are interviewed by the raters to determine at least one of whether the candidates are qualified subjects and to provide information to the raters.

16. The system of claim 15, wherein the at least one central rater site comprises at least one central rater site computer.

17. The system of claim 15, wherein, each of the at least one satellite site comprises at least one satellite site computer.

18. The system of claim 15, wherein the at least one central rater site is at least one of remote and centralized from the subjects.

19. The system of claim 15, wherein the at least one central rater site is at least one of a similar physical location and a virtual location.

20. The system of claim 15, wherein the raters use the at least one central rater site computer to interview the candidates at the at least one satellite site over a network, wherein the network couples the at least one satellite site to the at least one central rater site.

21. The system of claim 20, wherein the candidates receive interview questions and respond to interview questions using the at least one satellite site computer.

22. The system of claim of claim 20, wherein the network comprises at least one of the Internet, an intranet, satellite, and telephonic communication means.

23. The system of claim 21, wherein the candidates receive interview questions and respond to interview questions using video telephonic means, audio telephonic means, video conferencing, on-line chatting, and electronic mail.

24. The system of claim 22, further comprising means for connecting the at least one central rater site computer and the at least one satellite site computer to the Internet by high-speed connections.

25. The system of claim 22, further comprising means for enabling candidates at the at least one satellite site to communicate with raters at the at least one central rater site by communication between the at least one satellite site computer and the at least one central rater site computer over the network in at least one of real-time and almost real-time.

26. The system of claim 24, further comprising means for facilitating at least one of interactive streaming audio and interactive video media presentations presented through peripheral equipment of the at least one satellite site computer and the at least one central rater site computer.

27. The system of claim 15, wherein the at least one satellite site comprise at least one of a doctor's office, a clinic, a qualified subject's home, a hospital, a school, a factory floor, an office building, a shopping mall, and a house of worship.

28. The system of claim 15, further comprising a video-conferencing system to enable raters to interview candidates.

29. A computer-readable medium storing program code which, when executed on a computerized device, causes the computerized device to execute a method for centralized and standardized ratings of subjects in clinical trial studies, the method comprising:
training raters located at at least one central rater site to employ substantially similar criteria for at least one of recruiting qualified subjects and collecting information from the qualified subjects, wherein the at least one central rater site is at least one of a similar physical location or a virtual location; and
interviewing qualified subject by the raters located at the at least one central rater site, wherein the interviewing is to accomplish the at least one of recruiting qualified subjects and collecting information from the qualified subjects.

30. The computer-readable medium of claim 28, wherein the computer-readable medium further enables the interviewing to be accomplished utilizing communications over a network.

31. The method of claim 29, wherein the network comprises the Internet, an intranet, satellite, and telephonic communication means.

32. The method of claim 29, wherein the interviews are accomplished using at least one of video conferencing, audio conference, a live on-line chat, and electronic mail.
